(19) United States
(12) Patent Application Publication
(10) Pub. No.: US 2011/0190635 A1
(43) Pub. Date: Aug. 4, 2011

(54) ULTRASOUND SCANNER WITH GEL DISPENSER DEVICE ATTACHED TO OR INTEGRATED WITH AT LEAST ONE PROBE AND USE

(76) Inventor: Frédéric Bosler, Sarrebourg (FR)

(21) Appl. No.: 12/864,953

(22) PCT Filed: Jan. 9, 2009
(86) PCT No.: PCT/FR09/00030

§ 371 Date: Sep. 21, 2010

(30) Foreign Application Priority Data
Jan. 29, 2008 (FR) 0800475

(51) Int. Cl. A61B 8/00

(52) U.S. Cl. 600/458

(57) ABSTRACT

The invention relates to an ultrasound scanner comprising a gel distributing device which is attached, or built into, at least one signal emitting probe. The ultrasound scanner is provided with a gel holder for gel used to transmit ultrasound waves for medical, paramedical and/or veterinary procedure purposes. The holder is fixed to the probe and has an opening in the vicinity of the signal emitting or receiving window of the probe. The holder is a tight, sterile, supple pouch, which is tightly connected by tubes or the equivalent to a pump which, in turn, is tightly connected to at least one probe. The invention also relates to the use of the fluid circuit of the ultrasound scanner, comprising at least the tight, sterile, supple pouch that is tightly connected via the tube to the pump set which, in turn, is tightly connected via the tube for supplying the signal emitting and/or receiving window of the probe with gel, in order to automatically prevent the operator from having to manually supply the gel and to stop the gel remaining for subsequent procedures from coming into contact with the air, the patient, or any other sources of contamination.

### BACKGROUND OF THE INVENTION

This application is a §371 US National Phase Patent Application of International Patent Application No. PCT/FR2009/000030, with an International Filing Date of Jan. 9, 2009, which claims priority to French patent application Ser. No. 08/00475, filed Jan. 29, 2008, which applications are hereby incorporated herein in their entirety and from which priority is hereby claimed under 35 U.S.C. §120.

This invention concerns medical, paramedical and veterinary ultrasound and so-called Doppler effect imaging such as, by way of example and used here for the description, an ultrasound scanner with at least one probe and for which an interface of the type gel suited to the transmission of ultrasound waves is an unavoidable requirement in order for the machine to be operational. The image is that of the body, for the purposes of the analysis of the movements, internal masses, measurements, etc. of the features targeted by comparison between the signals transmitted and those reflected back to the probe.

First of all, the invention relates to an ultrasound scanner.

Ultrasound scanners exist with a gel dispenser device attached to or integrated with at least one signal transmitting and/or receiving probe, including a reservoir of gel suited to the transmission of ultrasound waves for the purposes of medical, paramedical and veterinary procedures. This gel comes from a reservoir integral with the probe and is delivered by at least one orifice close to the signal transmitting and/or receiving window of the probe transmitting and/or receiving the said signals.

In the current state of the art, such fixed or portable ultrasound scanners include one or more probes connected by a signal transmission lead to the central processing unit, with a keyboard for control and setting purposes and various peripherals such as a screen, printer, this without limitation.

Each probe is equipped with an ultrasound signal transmitting and/or receiving window. The probes are convex in shape for low frequency transmitting and/or receiving for procedures involving targets situated deep inside the body, linear in shape for high frequency transmitting and/or receiving for targets situated close to the signal transmitting and/or receiving window and micro-convex in shape for transmitting and/or receiving at intermediate frequencies for targets situated between the deep and the superficial targets. Ultrasound scanners of this type are known as three-channel or n-channel scanners.

These procedures or examinations are performed by placing the signal transmitting and/or receiving probe in contact with the outer surface of the body concerned by the procedure directly above or close to the target. Such an operation can only take place if the operator applies between the transmitting and/or receiving window and the outer surface of the body being examined, a gel suited to the transmission of ultrasound waves. A right-handed operator must manipulate the probe in his right hand and at the same time use the controls on the peripherals and the keys on the keyboard with his left hand. The performance and the quality of the result depend directly on the synchronization of these actions.

In the current state of the art, the gel is contained in a bottle that the practitioner must handle separately. This manipulation is necessarily separate from the synchronous operations involved in performing the examination. Bottles of gel come in standard sizes of 250, 500 or 1000 milliliters suited to the characteristics and frequency of the examinations performed. For example, and by way of an approximation, 250 ml is enough for twenty examinations and/or one day's practice. For reasons due to the hygiene rules and the performance of the gel, it is not recommended that a bottle that has been opened and/or soiled by contact between its nozzle and the surface of the body examined be kept, or that the bottle be filled outside of a sterile environment. However, five-liter containers are in common use for the purposes of the refilling of bottles by the operators. The result of the foregoing is that the operator is faced with multiple obligatory constraints not only for the obtaining of a quality of information necessary to an effective, high-quality examination, but also for the performance of his equipment and for the efficiency and urgency of the examinations. Indeed, the results and the optimization of their characteristics depend directly on and are proportional to the homogeneity and regularity of the layer of gel placed between the signal transmitting and/or receiving window and the outer surface of the body being examined.

The problem posed by the prior art is illustrated by and focused on the bottle of gel. Indeed, the latter affects and pollutes the examination at every level, as described below and in this order: the equipment, the operator, the operating procedure, the patient, the environment and the immediate area.

Earlier ideas for automating the gel production operation on the areas examined and therefore for overcoming these disadvantages have not led to the realization of concrete, effective solutions.

As well as the previous observations, from the point of view of the equipment, it should be noted that the appearance of three or n-channel ultrasound scanners multiplies the problems below even further. A bottle containing gel must be uncapped, shaken if it has already been used, turned over to place the nozzle over the area to be examined, whilst avoiding touching the subject examined, and then be recapped for reasons of maintaining the sterility of the gel and the operation. Mission virtually impossible, for the flexible wall of the bottle pressed to express the gel sucks in the gel from the nozzle in contact with the external environment, and often accidently other things. A type of packaging such as the existing one will never be ideal due to the very fact of the variety of the characteristics of the examinations. A gel dries, soils or can become soiled and production of gel useful to the examination must necessarily be renewed. The packaging and the ambient temperature affect the viscosity of the gel and this can adversely affect the results. The larger the volume of the packaging, the longer the gel will take to reach the ambient temperature. The properties of viscosity and homogeneity are affected, and therefore also the results.

From the point of view of the operator, the bottle interferes with his concentration and the many manipulations temporarily or durably, but in any case surely and certainly affect the integrity of the functions of his hand, even his whole arm, as he must act repeatedly and frequently on the bottle.

From an operational point of view, a quantity of gel is deposited by guesswork before applying the signal transmitting and/or receiving window to the layer of gel thus constituted, which is then swept over by the probe, causing it to collect in heaps and inevitably soiling the equipment and the area examined. In the case of multiple interventions simultaneous with the ultrasound scanning, such as invasive medical procedures, there is a real risk of contamination and endangering the physical integrity of the patient. The time factor, which by deduction from the foregoing, is capital for the success of the operation, is indisputably increased by the use of gel in bottles. It should be noted that the operational and economic efficiency of these procedures is significantly affected.

From the patient's point of view and apart from the risk of contamination and/or the endangering of his physical integrity, there is also the question of the discomfort generated by the temperature of the gel, which makes the patient shiver, which for certain procedures may have an adverse effect on the result or lead to unnecessary repetition of the procedure.

Finally, from the point of view of the environment, and more particularly regarding the sterilization required for certain medical procedures or laboratory examinations, and as observed in the foregoing, the current packaging is incapable of meeting these requirements.

The aim of the invention is to take into consideration fully all these aspects and problems and to respond to them with a global, single solution using concrete and economically viable means.

Advantageously, the aim of this solution is to allow the performance of an ultrasound procedure in one smooth operation, continuously and effectively, whilst meeting all the requirements on hygiene and sterile operating procedures recommended and/or desirable in this field.

The present invention materializes everything that in spite of twenty years' application it has not possible to achieve for technical reasons relating to the formulation of an appropriate gel, the perfecting of a gel concentration and homogeneity suited to automated use, which requires at once the balancing of the pressure of the gel as it emerges from the pouch, the forces of suction by the pump, the delivery pressure of the pump and the diameter as well as the length of the different tubes, sleeves and nozzles in order to guarantee the consistency of the pressures in the different parts of the circuit on the one hand, and, on the other hand, a regular flow without turbulence so as to guarantee a constant flow rate on each delivery of gel through the orifice at the end of the circuit close to the signal transmitting and/or receiving window. Therefore the invention has had to take account of the ergonomics of existing devices so as not to modify them and not to interfere in any way with the operating protocols and procedures, whilst matching the flexibility of the tube materials with the pressure and fluidity criteria. In the end the invention makes it possible to guarantee the depositing in a precise location of a layer of homogeneous gel of a constant, calibrated thickness between the signal transmitting and/or receiving window and the outer surface of the body being examined. The automation of the said gel delivery implies, moreover, the lack of any defect in the tightness for the purpose of guaranteeing the sterility of the gel at all times, which implies connections that are at once perfectly tight and secure. And, once again in order to enable the application of the device to all ultrasound scanners available on the market as well as to all those being manufactured, the invention enables its application to multiple-channel ultrasound scanners and/or therefore to single-channel machines.

### SUMMARY OF THE INVENTION

Consequently, to solve these problems, the ultrasound scanner according to the invention is characterized in that the reservoir is a hermetic, sterile flexible pouch connected hermetically by tubes or equivalent to a pump itself connected hermetically to at least one probe.

The object of the invention is also the use of a circuit of fluid consisting of at least one sterile, hermetic, flexible pouch, connected hermetically via a semi-rigid tube to a pump assembly connected hermetically to a semi-rigid tube over at least a part of its length to deliver gel to the signal transmitting and/or receiving window of an ultrasound scanner probe so as to automatically spare the operator the parasitic action of manually delivering gel and to protect the remaining gel for future procedures against contact with the air, the patient or any other sources or factors of contamination.

### BRIEF DESCRIPTION OF THE DRAWINGS

The invention will be better understood by reading a non-limitative example of the embodiment of the present invention, which will now be described with reference to the figures enclosed:

FIG. 1 shows the ultrasound scanner according to the invention including a gel distribution circuit with a flexible gel reservoir pouch connected by tubes to a pump and a signal transmitting or receiving probe.

FIG. 2 shows the pump of the ultrasound scanner shown in FIG. 1.

### DETAILED DESCRIPTION

In the following detailed description of exemplary embodiments of the invention, reference is made to the accompanying drawings, which form a part hereof, and in which is shown by way of illustration specific exemplary embodiments in which the invention may be practiced. These embodiments are described in sufficient detail to enable those skilled in the art to practice the invention, and it is to be understood that other embodiments may be utilized and that logical, mechanical, electrical and other changes may be made without departing from the spirit or scope of the present invention. The following detailed description is, therefore, not to be taken in a limiting sense, and the scope of the present invention is defined only by the appended claims.

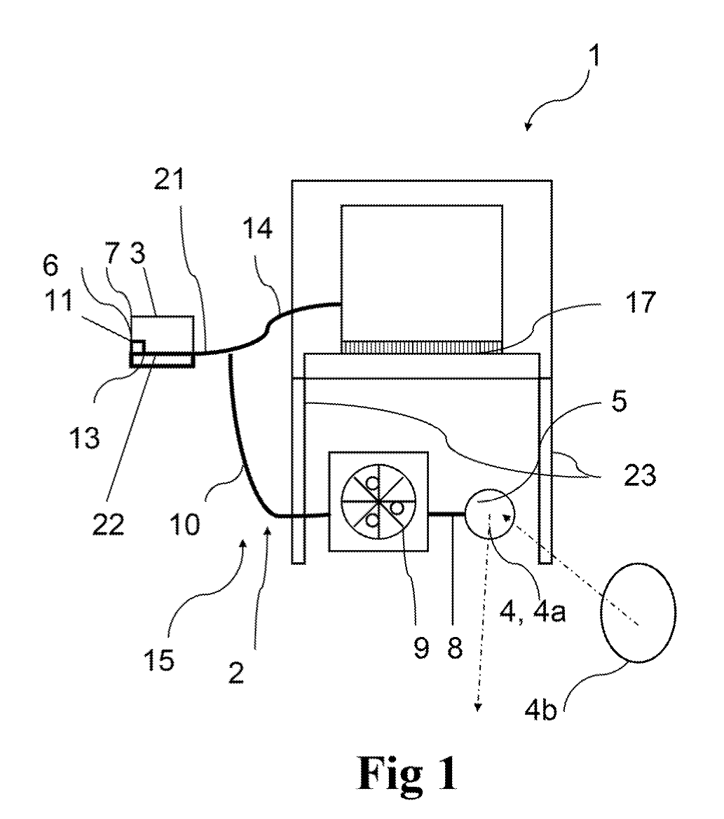

The ultrasound scanner 1, shown in FIG. 1, with a gel dispenser device 2 attached to or integrated with at least one signal transmitting and/or receiving probe 3, includes a reservoir of gel suited to the transmission of ultrasound waves for the purposes of medical, paramedical and/or veterinary procedures. The reservoir 4 integral with the probe 3 contains gel 5 which is delivered through at least one orifice 6 close to the signal transmitting and/or receiving window 7 of the probe 3. The reservoir represents a sterile, hermetic, flexible pouch 4a. The said pouch 4a is connected hermetically by means of delivery such as tubes 8 or equivalent to a pump 9 itself connected hermetically to at least one probe 3.

The flexible pouch 4 has walls thin enough to follow the form of the gel as the latter is drawn off from the pouch by the pump 9 and under the effect of the ambient atmospheric pressure, without altering the suction force of the said pump 9. The shape-following properties of the pouch 4a and the flexibility of its walls provide a large surface area allowing a rapid and significant heat exchange, allowing the temperature of the gel to be brought to the ambient temperature. In another variant of the embodiment, the said pouch 4a may, if necessary, be lodged in a housing integral with the pump containing devices for warming the gel to the comfortable ambient temperature.

The gel 5 dispenser device 2 attached to or integrated with at least one signal transmitting and/or receiving probe 3, includes the sterile, hermetic, flexible pouch of gel suited to the transmission of ultrasound waves for the purposes of medical, paramedical and/or veterinary procedures. Which pouch 4a is removable and replaceable by any other pouch 4b of the same type and capacity suited to the characteristics of the procedure performed, and typically calibrated at 250, 500 or 1000 milliliters. In fine the invention enables the ideal volume required for series of standard procedures to be used.

The pouch 4a, 4b, the pump 9 and the different tubes 8, 10 through which the gel 5 flows form a sterile, hermetic circuit with a single opening 11 at the transmitting and/or receiving window 7 and more particularly in which the pump 9 is a peristaltic pump.

The flow tubes are mainly of two categories: the first includes the tube 8 which draws the gel from the pouch 4a, 4b and carries it to the pump 9, the means of interconnection (tube(s), sleeve(s), openings not visible in the figures) of the pump with the multichannel drawer 12 that distributes the gel to the connection nozzles 13 of each of the channels corresponding to each type of probe equipping the ultrasound scanner 1. The second includes the tube delivering the gel to the probe The first category in the preferred embodiment of the invention is a tube 8 with a cross section of 4 mm and a length of 200 mm maximum before the pump 9 and for after the pump a tube 10 of unlimited length, but corresponding to the length of the signal transmission lead 14 in use. The second category will have a cross section of 4 mm, but be of unlimited length much greater than those of the first category.

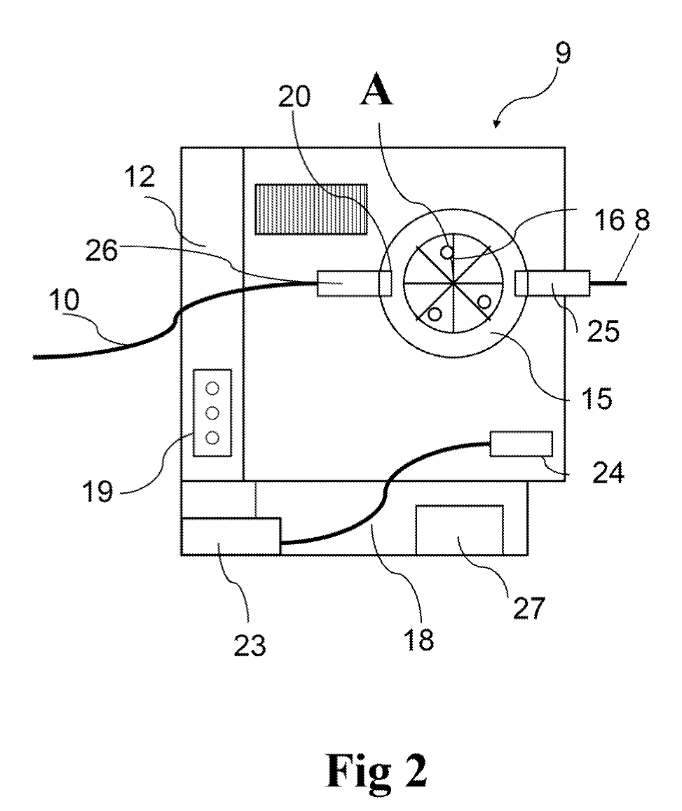

The peristaltic pump 9, shown in FIG. 2, consists of a sealed housing 15 with a transparent cover through which the flexible tube of the pump 9 can be seen, compressed in turn by, but without being limited to this, at least two rollers 16 out of three arranged on a plate in the shape of a star with three arms integral with a rotating shaft A emerging through the bottom of the housing 15 of the pump 9 via a sealed self-lubricated bearing, which shaft A connects to an electric motor of a known type, not shown, connected to an electricity supply terminal block via an electric circuit make/break contactor. The contactor is connected to an actuation device such as a foot control and/or control button which can be placed anywhere, such as advantageously and non-exclusively on the keyboard 17 of the ultrasound scanner 1 and/or again a touch sensitive control such as, but without being limited to that, a Zener diode type control, and the said flexible tube in the pump connects to two connection nozzles, one for the tube 8 drawing the gel from the pouch and the other for, depending on the embodiment, the tube 10 delivering the gel to the probe 3 or to the interconnection tubes not visible on the figure of the pump 9 with the multichannel gel distribution drawer 12.

The peristaltic pump 9 is designed to meet the criteria selected concerning the flow and pressure of the gel. These criteria may, in another embodiment, be influenced by a device varying the speed of rotation of the motor shaft in order to enable the optimization of the results of certain ultrasound procedures.

The pump 9 is rendered integral, as well as its peripheral equipment, with a casing 18 containing the pump 9, the said casing 18 being able without limitation, according to needs, to receive all the alarm, recognition, identification, reading, measurement, management devices, manual, programmable and automated and relating to the probe or probes, the characteristics of the flow, those of the gel, the external temperature and that of the gel, the operating time, the volumes, the frequencies, the heating of the gel, the maintenance, and in fine to those concerning the pump 9.

Thus, advantageously, the peristaltic pump is not in contact with the gel. No backflow of gel is possible, therefore no backflow of gel from the probe.

The multichannel distribution drawer 12 consists of a case inside which there is a mobile, sliding or rotating, drawer, forming a sealed assembly, the drawer including internal channels so that one of the ends of any of the channels meets the end of the tube 8 drawing off the gel from the pouch and the other end of the same channel meets the end of the tube 10 delivering gel to the probe, the movement of the drawer being able to be mechanical and manually or electrically controlled, the distribution drawer being able to be a succession of sealed solenoid valves 19, the electric controls of the drawer or of the solenoid valves 19 being able to be assisted electronically by electrical and/or magnetic impulses received from the appropriate probe used for the ultrasound procedure envisaged.

The pouch 4a, 4b contains a gel formulated with a concentration suited to the optimization of the performances of the ultrasound scanner 1 as a whole with one third standard gel available on the market and two thirds sterile water.

The invention as described offers with the potential equipment in the casing 18 containing the pump 9, a means of electronically controlling the pump 9, the concentration and homogeneity of the gel, the pressure and speed of flow of the gel, and allows the formulation of the gel to be modified in order to allow the optimization of the results of certain ultrasound procedures.

The pouch 4a, 4b contains a cleaning and sterilizing solution suited at least to the requirements of servicing and placing the circuit on prolonged standby. This possibility provides a further guarantee of the correct, sterile operation of the ultrasound scanner 1 and prevents any deposits that could create turbulence or interference with the flow of gel following the placing of the devices on prolonged standby or a significant stoppage or exogenous phenomena, such as for example substantial changes in the temperature leading to structural changes in the gel.

The gel is permanently and lastingly in contact with and enveloped by the flexible membrane of the said pouch 4a, 4b.

All the connections 28 of the tubes 8, 10 in the gel supply circuit are secure, sterile, sealed connections 20, typically so-called push-pull connectors or with devices of the medical puncture needle type.

The connection of the tube 8 drawing off the gel from the flexible pouch 4a, 4b is of the medical puncture needle type. This device is known for being used with blood transfusions pouches and is used whenever sterility and airtightness are a requirement.

All the other connections 20 in the invention are of the type with a male end piece able to penetrate a female piece by pushing for a first engagement, thus making a solid, secure and airtight connection which can only be undone by a second firm pushing of the male end piece as far as it will go inside the female piece in order to release the locking device whilst compressing the connection 20 so as to be able, in a second consecutive operation, to pull on the female piece so as to separate the two pieces. This type of connection 20 is known as a push-pull connector. The invention gives preference to these connectors due to their performance and reliability, but they are not compulsory.

The tube 10 delivering the gel to the probe 3 is either rendered integral with the lead 14 transmitting the signals to the central processing unit by a medical shape memory or self-retracting sleeve 21, creating a single hermetic, sterile assembly, or is an integral part of the signal transmission lead 14. In the first eventuality, this solution is suitable for any ultrasound scanner 1 distributed or in use. The second eventuality applies to the manufacturing of signal transmission leads 14 in the factory. For the needs of maintenance, it is necessary to provide a way of replacing the tube 10 delivering the gel to the probe 3. To this effect, the invention provides that the signal transmission lead 14 form a concentric accumulation of cables with the tube 10 delivering the gel to the probe 3 centered in this assembly so that it can easily be slid. Another possibility is that the signal transmission lead 14 consist of a sealed, hermetic shell-like sheath 21 containing at once the signal transmission lead 14 and the tube 10 delivering the gel to the probe 3, which sheath 21 may, if necessary, open along its length to remove the tube 10 delivering the gel to the probe 3.

The tube 10 delivering the gel to the probe 3 is semi-rigid to preserve the flexibility of the signal transmission lead 14 and keep the pressure and flow of the gel constant and identical to those of the gel as it emerges from the peristaltic pump 9. Indeed, it is on this semi-rigidity that the quality of the metering of the volume of the gel at the signal transmitting and/or receiving window 7 depends. An optimization can then be obtained using the controls and systems in the casing 19 containing the pump 9 and/or the gel formulation.

The semi-rigid tube 10 delivering the gel to the probe 3 includes at the opposite end to the pump a flexible adapter 22 delivering the gel onto the signal transmitting and/or receiving window 7 of the probe, so as to preserve the usual flexibility and the operational mobility of the probe. Indeed, the degree of flexibility of the signal transmission lead 14 is much lower than the degree of flexibility at the connection with the probe 3. The accuracy of the ultrasound procedure depends on this flexibility. Only a flexible material is suitable. Advantageously the flexible material of such a tube does not hinder the movements of the person manipulating the probe 3 as it is flattened by the adherence of almost half of its external surface circumference on the body of the probe. This flattened appearance in no way modifies the quality and performance of the gel flows at the signal transmitting and/or receiving window 7.

The use of a circuit of fluid consisting of at least one sterile, hermetic, flexible pouch 4a, 4b, connected hermetically via a semi-rigid tube 8 to a pump assembly 9 connected hermetically to a tube 10 semi-rigid over at least a part of its length to deliver gel to the signal transmitting and/or receiving window 7 of a probe 3 of an ultrasound scanner as described previously enables the operator to be automatically spared the parasitic action of manually delivering gel and the remaining gel to be protected for future procedures against contact with the air, the patient or any other sources or factors of contamination

Although the invention has been described with reference to particular structures, it is in no way limited to them and numerous variants may be employed.

The combinations of the different embodiments represented in the drawings or described above do not fall outside the scope of the invention.

### REFERENCE ITEMS

1. ultrasound scanner
2. gel dispenser device
3. signal transmitting probe
4. reservoir
4a. flexible pouch
4b. flexible pouch
5. gel
6. orifice
7. transmitting window
8. tube
9. pump
10. tube
11. single opening
12. multichannel drawer
13. nozzle
14. signal transmission lead
15. housing
16. rollers
17. control and setting keyboard
18. casing containing the pump
19. solenoid valves
20. connections
21. sleeve or sheath
22. flexible gel delivery adapter
23. actuation device
24. circuit make/break contactor
25. connection nozzle
26. connection nozzle
27. alarm, recognition, identification, reading, measurement, management devices, manual, programmable and/or automated

The references inserted after the technical characteristics mentioned in the claims are included with the sole aim of facilitating the comprehension of the latter and in no way limit their scope.

While the foregoing detailed description has described several embodiments of this invention, it is to be understood that the above description is illustrative only and not limiting of the disclosed invention.

1. Ultrasound scanner comprising a gel dispenser device attached to or integrated with at least one signal transmitting probe that includes a signal transmitting and/or receiving window, the probe further including a reservoir of gel suited to the transmission of ultrasound waves for the purpose of medical, paramedical and veterinary procedures, which gel comes from a reservoir integral with the probe delivered through at least one orifice close to the signal transmitting and/or receiving window the reservoir comprising a sterile, hermetic flexible pouch connected hermetically by at least one tube to a pump that is connected hermetically to the at least one probe.

2. Ultrasound scanner according to claim 1, wherein the pouch is configured to be removable and replaceable by any other pouch of the same type and capacity that is suited to a procedure to be performed.

3. Ultrasound scanner according to claim 2, wherein the pouch is calibrated at one of about 250 millimeters, about 500 millimeters and about 1000 millimeters.

4. Ultrasound scanner according to claim 1, wherein the pouch, the pump and the at least one tube through which the gel flows form a sterile, hermetic gel supply circuit with a single opening at the transmitting and/or receiving window, wherein the pump is a peristaltic pump.

5. Ultrasound scanner according to claim 4, wherein the peristaltic pump includes a sealed housing with a transparent cover through which the at least one tube of the pump can be seen, compressed in turn by at least two rollers out of three arranged on a plate in the shape of a star with three arms integral with a rotating shaft emerging through the bottom of housing of the pump via a sealed self-lubricated bearing, which shaft connects to an electric motor connected to an electricity supply terminal block via an electric circuit make/break contactor, the contactor being connected to an actuation device, the at least one tube in the pump connects to two connection nozzles, one connection nozzle for a tube drawing the gel from the pouch and another connection nozzle for the at least one tube delivering gel to the probe or indirectly via the tubes in the pump with a multichannel gel distribution drawer and with a device configured to vary a speed of rotation of the motor shaft.

6. Ultrasound scanner according to claim 5, wherein the actuation device includes one of a foot control, a control button, and a Zener diode type touch sensitive control.

7. Ultrasound scanner according to claim 5, wherein at least the pump is rendered integral with a casing containing the pump, the casing being configured, at least, to receive all the alarm, recognition, identification, reading, measurement, management devices, manual, programmable and automated and relating to the probe or probes, characteristics of the flow, characteristics of the gel, an external temperature and a temperature of the gel, an operating time, volumes, frequencies, heating of the gel, and maintenance of the pump.

8. Ultrasound scanner according to claim 5, wherein the multichannel distribution drawer includes a case inside which there is provided a mobile, sliding or rotating, drawer, forming a sealed assembly, the drawer including internal channels so that one of the ends of any of the channels meets an end of the tube that draws the gel from the pouch and another end of the same channel meets an end of the tube that delivers gel to the probe, the movement of the drawer being configured to be mechanical and manually or electrically controlled, the distribution drawer including a succession of sealed solenoid valves 19, electric controls of the drawer or of the solenoid valves being assisted electronically by electrical and/or magnetic impulses received from the probe.

9. Ultrasound scanner according to claim 1 further comprising a gel dispenser device attached to or integrated with the at least one signal transmitting probe, wherein the pouch contains a gel formulated with one third gel and two thirds sterile water.

10. Ultrasound scanner according to claim 4, wherein the pouch contains a cleaning and sterilizing solution suited at least to requirements of servicing and placing the gel supply circuit on prolonged standby.

11. Ultrasound scanner according to claim 1, wherein the pouch includes a membrane that is flexible, that maintains contact with the gel and that envelops the gel so as to avoid contact of the gel with the ambient air.

12. Ultrasound scanner according to claim 4, wherein all connections of tubes that supply the gel supply circuit are secure, sterile, sealed connections, of one of a push-pull connectors type and of a medical puncture needle type.

13. Ultrasound scanner according to claim 5, further comprising a signal transmission lead and wherein the tube that is configured to deliver the gel to the probe is one of rendered integral with the signal transmission lead by a medical shape memory or self-retracting sleeve creating a single hermetic, sterile assembly, and is an integral part of the signal transmission lead.

14. Ultrasound scanner according to claim 5, wherein the tube that is configured to deliver the gel to the probe is semi-rigid to preserve the flexibility of the transmission lead and keep the pressure and flow of the gel constant and identical to those of the gel as it emerges from the peristaltic pump.

15. Ultrasound scanner according to claim 14, wherein the semi-rigid tube that is configured to deliver the gel to the probe includes at an opposite end to the pump a flexible adapter that is configured to deliver the gel onto the signal transmitting and/or receiving window of the probe, so as to preserve a flexibility and operational mobility of the probe.

16. Use of a fluid circuit comprising at least one sterile, hermetic, flexible pouch, connected hermetically via a semi-rigid tube to a pump assembly connected hermetically to a tube that is semi-rigid over at least a part of its length to deliver gel to the signal transmitting and/or receiving window of a probe of an ultrasound scanner according to claim 1 to automatically spare the operator from having to manually deliver gel and to protect remaining gel for future procedures against contact with the air, the patient or other sources of contamination.
